(19) United States
(12) Patent Application Publication
(10) Pub. No.: US 2023/0310316 A1
(43) Pub. Date: Oct. 5, 2023

(54) Brain Targeted Nutritional Therapeutic for Improved Cognitive Function and Treatment of Mild Cognitive Impairment

(71) Applicant: BRAINCARE LLC, Reno, NV (US)

(72) Inventor: Charles H. Marsland, Windermere, FL (US)

(21) Appl. No.: 18/206,895

(22) Filed: Jun. 7, 2023

Related U.S. Application Data
(63) Continuation of application No. PCT/US21/62149, filed on Dec. 7, 2021.
(60) Provisional application No. 63/122,158, filed on Dec. 7, 2020.

(51) Int. Cl. A61K 9/00; A61K 31/198; A61K 31/352; A61K 31/4188; A61K 33/18; A61K 31/525; A61K 33/08; A61K 31/385; A61K 31/375; A61K 31/593; A61K 31/714; A61P 25/28

(52) U.S. Cl. CPC A61K 9/0095; A61K 31/198; A61K 31/352; A61K 31/4188; A61K 33/18; A61K 31/525; A61K 33/08; A61K 31/385; A61K 31/375; A61K 31/593; A61K 31/714; A61P 25/28

(57) ABSTRACT

There are no medical treatments available for cognitive decline in humans. Prevention through improved lifestyle, especially related to diet, is the one way to reduce the risk. Paradoxically, the modern diet is one that increases dementia risk and worsens cognitive function. Therefore it is critical that new methods be developed based on nutrient therapy.

### CROSS-REFERENCE TO RELATED APPLICATION

This application is a continuation of PCT/US2021/062149, filed Dec. 7, 2021, which claims priority of Provisional Patent Application Ser. No. 63/122,158 filed on Dec. 7, 2020, the disclosure of which is incorporated herein by reference.

### BACKGROUND

This disclosure relates to a nutritional therapeutic.

Cognitive Impairment and Dementia are the greatest global challenge for health and social care in the 21st century. It occurs mainly in people older than 65 years, so the increases seen in numbers and costs are driven by increased longevity. No validated treatments are available so prevention is the only way to slow the incidence of cognitive impairment and dementia.

Besides age, modifiable risk factors for dementia are mostly related to lifestyle with diet and exercise having the greatest impact. Diet-related chronic conditions like cardiovascular disease and type 2 diabetes mellitus also increase risk. Adopting a healthy lifestyle can reduce risk of dementia even in those with a high genetic risk profile. A healthy lifestyle in genetically-predisposed individuals is reported to have only a 1.13% risk compared to those at risk who did not adopt a healthy lifestyle, who had a risk of 1.78%.

Up until 2010, scientists at the National Institutes of Health and other prominent institutes all but dismissed diet as having any role in preventing Alzheimer's disease. However, eight years later, 109 scientists from 36 countries wrote a letter to the G8 Dementia Summit in London, stating that enough evidence exists to support a role for consuming a healthy, nutrient-dense diet because it reduces the risk of Alzheimer's disease. Other risk factors identified in this correspondence included maintaining a healthy body weight, not smoking, and avoiding hypertension. Adoption of these factors can reduce the risk of dementia by 35%, which has more of an impact than genetic risk factors.

Dietary modifications to the reduce risk of dementia have been studied extensively and the results are remarkably consistent. Adopting healthy food regimens such as the Mediterranean Diet and the MIND Diet have been shown to improve cognitive function and reduce dementia risk in an aging population. The MIND Diet reduced cognitive decline by the equivalent of 7.5 years. Others have shown that both diets are likely to be effective because they are rich in fruits and vegetables; low in saturated fats; and high in polyunsaturated fats and are high in nutrients plus polyphenols. Despite these positive findings on what dietary patterns to adopt to slow cognitive decline, most people do not consume foods that are found in the Mediterranean Diet or MIND Diet. Specifically related to cognition, people everywhere eat too few fruits and vegetables, too many foods rich in all fats, and do not get enough of the essential nutrients.

In addition to the dietary components, bioactive compounds have shown promise in enhancing cognitive function. Phosphatidylserine (PS) supports many aspects of human cognitive function including: short-term memory, long-term memory, creating new memories, retrieving old memories, learning and recalling information, focusing, concentrating, reasoning and problem solving, and communicating skills. Two naturally occurring compounds not ordinarily consumed in the diet, acetyl-L carnitine (ALC) and alpha-lipoic acid (ALA), are highly effective antioxidants. Both ingredients protect brain cells from oxidation, allowing for maintenance of healthy energy production at a cellular level. N-acetyl cysteine (NAC) is a potent antioxidant offering protection to the brain cell mitochondria, thereby supporting memory. Epidemiological studies indicate that consumption of quercetin may lower the relative risk for Alzheimer's dementia. Coenzyme Q-10 may protect against mitochondrial brain cell damage. Brain mitochondria have antioxidant defenses inside the cell, making mitochondrial DNA 10-100 times more likely to become damaged than nuclear DNA. The exact mechanism of how antioxidants protect against mitochondrial damage is unknown, but oxidation is a universal contributor to all neuro-degenerative disorders.

The invention highlights the validated and clinically proven effects of this novel brain targeted nutritional therapeutic beverage with proprietary chemistry directed on cognitive function in humans. This new strategy is needed to prevent cognitive impairment and decline that occurs with aging. Dietary interventions and forced change have shown promise but are difficult, force behavior change and are rarely adopted. An easy-to-prepare brain targeted nutritional therapeutic beverage shown to support the brain helps improve cognition is needed.

### SUMMARY

In one aspect, featured herein is a brain-targeted nutritional therapeutic beverage that improves cognitive function by at least 14% and up to 44% including forgetfulness, distractibility, and false triggering. In some examples the nutritional therapeutic beverage contains a composite mixture of L-Phenylalanine, L-Tyrosine, L-Arginine, N-Acetyl-L-Cysteine (NAC), Acetyl-L-Carnititine, Quercetin, Alpha Lipoic Acid, Coenzyme Q10 (Ubidecarenone). In an example the nutritional therapeutic beverage includes a mixture of at least Calcium, Magnesium, Manganese, Phosphorus, Potassium, Vitamin C, Vitamin D, Vitamin K, Biotin, Chloride, Chromium, Copper, Folate/folic acid, Molybdenum, Niacin, Pantothenic acid, Riboflavin, Selenium, Sodium, Thiamin, Vitamin A, Vitamin B6, Vitamin B12, Vitamin E, Zinc, Choline, Iodine, Iron, L-Phenylalanine, L-Tyrosine, L-Arginine, N-Acetyl-L-Cysteine (NAC), Acetyl-L-Carnitine, Quercetin, Alpha Lipoic Acid, Coenzyme Q10 (Ubidecarenone). In an example the nutritional therapeutic beverage includes a mixture of at least L-Leucine, L-Isoleucine, L-Valine, L-Tryptophan, L-Histidine, L-Lysine, L-Methionine, L-Threonine, L-Cysteine, Calcium, Magnesium, Manganese, Phosphorus, Potassium, Vitamin C, Vitamin D, Vitamin K, Biotin, Chloride, Chromium, Copper, Folate/folic acid, Molybdenum, Niacin, Pantothenic acid, Riboflavin, Selenium, Sodium, Thiamin, Vitamin A, Vitamin B6, Vitamin B12, Vitamin E, Zinc, Choline, Iodine, Iron, L-Phenylalanine, L-Tyrosine, L-Arginine, N-Acetyl-L-Cysteine (NAC), Acetyl-L-Carnitine, Quercetin, Alpha Lipoic Acid, Coenzyme Q10 (Ubidecarenone). In an example the nutritional therapeutic beverage also includes Phosphatidyl Serine. In an example the nutritional therapeutic beverage also includes the chelated minerals: Copper Bisglycinate Chelate, Zinc Citrate Dihydrate, and Potassium Gluconate, as well as Phosphatidyl Serine L-Leucine.

In a specific example a single serving of the nutritional therapeutic beverage includes the following ingredients and amounts of ingredients:

| Ingredient | wt. % |
|---|---|
| L-Leucine | 1.5654% |
| L-Isoleucine | 0.8028% |
| L-Valine | 1.0436% |
| L-Tryptophan | 0.1606% |
| L-Histidine | 0.5396% |
| L-Lysine | 1.5046% |
| L-Methionine | 0.4014% |
| L-Threonine | 0.6021% |
| L-Cysteine | 0.2316% |
| L-Phenylalanine | 0.5017% |
| L-Tyrosine | 0.5017% |
| L-Arginine | 0.2649% |
| N- Acetyl -L-Cysteine | 1.3042% |
| Acetyl-L-Carnitine Hydrochloride | 0.5968% |
| Phosphatidyl Serine | 1.5814% |
| Quercetin | 0.4658% |
| Alpha Lipoic Acid | 0.1597% |
| Coenzyme Q10 (Ubidecarenone) | 0.0807% |
| Copper Bisglycinate Chelate | 0.0104% |
| Zinc Citrate Dihydrate | 0.0250% |
| Potassium Gluconate | 1.5395% |
| Tripotassium Citrate | 0.7106% |
| Calcium Lactate | 4.0995% |
| Chromium Chloride Hexahydrate | 0.0003% |
| Microencapsulated MgO | 1.0872% |
| Manganese Sulfate 1 H2O USP/FCC Powder | 0.0033% |
| Potassium Iodide USP | 0.0001% |
| Dipotassium Phosphate | 2.9958% |
| Selenium L-Methionine Complex | 0.0076% |
| Molybdenum Trituration | 0.0008% |
| Vit A Palmitate | 0.0179% |
| Thiamin Mononitrate | 0.0019% |
| Cyanocobalamin | 0.0005% |
| Riboflavin | 0.0015% |
| Niacinamide | 0.0154% |
| D-calcium Pantothenate | 0.0105% |
| Pyridoxine HCL | 0.0019% |
| D-Biotin | 0.0219% |
| Folic Acid | 0.0036% |
| Microencapsulated Ascorbic Acid | 0.1023% |
| Microencapsulated Vit D 3 Powder | 0.0033% |
| DL α-tocopheryl acetate | 0.0438% |
| Vit K1 | 0.0067% |
| Maltodextrin | 0.0138% |
| Choline Bitartrate | 0.7195% |
| Palm Kernel Oil | 0.1281% |
| Oat Fiber | 0.1020% |
| Dry Milk | 14.2326% |
| Whey Protein Concentrate 80 | 17.3954% |
| Sea Salt | 0.1581% |
| Fiber | 3.9535% |
| Vanilla | 1.1861% |
| Bitter Blocker | 0.1265% |
| Cocoa Powder | 12.6512% |
| Cream Powder | 3.1628% |
| Guar Gum | 1.1861% |
| Flax Seed | 3.1628% |
| Sugar | 6.0093% |
| Monk Fruit | 0.0633% |
| Stevia | 0.0633% |
| Non- GMO Expeller Pressed Canola Oil | 12.6512% |
| Ferrous Bisglycinate Chelate | 0.0144% |
| Total | 100% |

### BRIEF DESCRIPTION OF THE DRAWING

The drawing is a plot of individual changes in Subjective Memory questionnaire total scores.

### DETAILED DESCRIPTION

To demonstrate and validate the effectiveness of the novel brain targeted nutritional therapeutic, a single-arm study was conducted to explore the effects of a brain targeted nutritional therapeutic beverage on cognitive function in humans. Seven individuals (86% females; 59±6 years), who stated they were worried about their cognition, completed the 6-week study and consumed two of the brain targeted nutritional therapeutic beverages daily. At baseline, week 3, and week 6, cognitive function measurements were obtained using two medically qualified surveys (Cognitive Failures and Subjective Memory Questionnaires); and quality of life information was obtained weekly.

Results: All summary measures of Cognitive Failure Questionnaire improved after 3 weeks, and again more at week 6. The Subjective Memory Questionnaire revealed the mean baseline total score was representative of a group that had a modest appreciation of losing memory. Quality of life questions (general feeling of wellbeing, fullness, energy, sleep, appearance, and diet quality) improved by 67%.

Conclusion: Use of two brain-targeted nutritional therapeutic beverages daily improved cognitive function and quality of life in older individuals who were worried about maintaining their brain function.

Methods

To determine the magnitude of effects on humans, the nutritional therapeutic was put into a single-armed, prospective study. Baseline data was obtained using two cognition questionnaires, dietary habit information, quality of life questions, exercise routine information, and anthropometry. Subjects then began consuming two brain-targeted nutritional therapeutic beverages per day for 6-8 weeks. Weekly data was monitored for compliance. At weeks 3 and 6, additional cognitive testing using the same surveys occurred. Data was stored in a HIPAA-compliant area and no participant was identified by name.

Participants

Subjects concerned with cognitive decline were recruited through social media (e.g., Facebook, Instagram). Entry criteria included: being age 50-70 years, having a Body Mass Index (BMI) 25-40 kg/m², having not been diagnosed with Alzheimer's disease or mild cognitive impairment (MCI), and may have a chronic condition like diabetes or high blood pressure. All participants signed a consent that abided by the Helsinki Declaration, seventh revision and willingly participated in the study. All participants were assigned a coach to help with dietary compliance and completing the electronically-submitted data collection forms that included information about anthropometry (body weight and waist circumference), and questions related to cognition and quality of life

Dietary Intervention and Anthropometry

Participants were provided two daily servings of a brain-targeted nutrition therapeutic beverage for 8 weeks. The product was provided as a powder, which could be reconstituted with water, milk, or a milk substitute (e.g., nut, soy). Each serving contained one-third of the Daily Value for every essential vitamin and mineral, except sodium and chloride (https://nutrientsurvival.com/). The Food and Drug Administration (FDA) has set Daily Values (i.e., how much people aged 4 years of age and older should consume daily) for essential vitamins and minerals (https://www.fda.gov/food/new-nutrition-facts-label/daily-value-new-nutrition-and-supplement-facts-labels). At least 25% Adequate Intakes (AI), defined by the FDA, for omega-3s and 33% of the AI for choline (370 mg; based on the need of males) was included in each serving (SOURCES: Dietary Reference Intakes for Calcium, Phosphorous, Magnesium, Vitamin D, and Fluoride (1997); Dietary Reference Intakes for Thiamin, Riboflavin, Niacin, Vitamin B6, Folate, Vitamin B12, Pantothenic Acid, Biotin, and Choline (1998); Dietary Reference Intakes for Vitamin C, Vitamin E, Selenium, and Carotenoids (2000); Dietary Reference Intakes for Vitamin A, Vitamin K, Arsenic, Boron, Chromium, Copper, Iodine, Iron, Manganese, Molybdenum, Nickel, Silicon, Vanadium, and Zinc (2001); Dietary Reference Intakes for Water, Potassium, Sodium, Chloride, and Sulfate (2005); and Dietary Reference Intakes for Calcium and Vitamin D (2011). These reports may be accessed via www.nap.edu.). Free nutrition coaching was provided to assist with questions about the dietary intervention and aid compliance. Each week, the participants reported how many of the two brain-targeted beverages they consumed and how much of each they drank.

Dietary information potentially related to cognitive function was obtained at baseline, including the frequency of intake of: red meat/cold cuts, alcohol, seed/nut intake, fruit and vegetables, fast food, sugar, and fish.

Participants provided body weight measurements weekly, and waist circumference readings at baseline, week 3, and week 6 (National Heart, Lung, and Blood Institute, 2020).

Cognitive Testing

Cognitive Failures Questionnaire (CFQ) (Cognitive Failures Questionnaire, 2020)

The CFQ probes minor mistakes that most people make occasionally but sometimes they happen more frequently. The survey was developed to assess the frequency that individuals experience these mistakes in cognitive function, such as absent-mindedness, in everyday life—slips and errors of perception, memory, and motor functioning. The CFQ scoring system used for each question was: 0=Never; 1=Very rarely; 2=Occasionally; 3=Quite often; 4=Very often. Summed sub-scale scores with similar attributes were also determined for a Total CFQ score; Forgetfulness (a tendency to forget something that is known or planned, for example, names, intentions, appointments, and words); Distractibility (mainly in social situations such as being absentminded or easily disturbed with one person or in a group); and False triggering (interrupted ability to pay attention leading to making errors in thinking and acting logically).

Subjective Memory Questionnaire (SMQ)

The Subjective Memory Questionnaire (SMQ) that includes 43 questions has been validated (McMillian T M, 1984; Bennett-Levy and Powell G E, 1980). However, we used a sub-set of these questions (i.e., 33) that specifically examine difficulty with memory (Learning Strategies, 2020). A sliding-scale scoring system 1 (Poor), 3 (Good), and 7 (Excellent) was used, with higher scores relating to fewer subjective memory difficulties. The sum of the 43 responses was scored as: 200+=minimal subjective memory difficulties; 100-200=noticing a moderate degree of memory challenge; and below 100=a greater self-awareness of memory difficulties.

Quality of Life Questions

At baseline and weekly, subjects answered nine questions about their quality of life. Each was rated on a scale of 1 to 5, with 5 being the best and 1 being the worst. The questions asked were: general feeling, fullness, mood, energy level, any gastrointestinal symptoms (GI), sleep quality, appearance, and diet quality. Data were compared at baseline, week 3, and week 6, and the percentage change between the mean scores at baseline and week 6 was calculated.

Statistics

Data are presented as means±standard deviations. Both t-test and paired t-test were used to determine significance of the cognitive function tests.

Results

Eight subjects started the study and seven completed it (88% retention). One male and six females participated with an average age of 59±6 years. The mean body mass index of the group was 30±5 kg/m². The male had a normal waist circumference (91 cm), but the mean waist circumference of the females exceeded the healthy limit of 88 cm (97±14 cm). Patients remained weight stable and no change occurred in waist circumference (data not shown). This was an objective of the study so weight change would not be a confounding variable to changes in cognitive function.

One participant had no co-morbidities, three had hypertension, and one each had these conditions: arthritis, small cerebral vessel ischemic disease, and prediabetes. Five required medications to treat these conditions.

Baseline Dietary and Exercise Information

All participants claimed that they were not provided dietary counseling by a healthcare professional about preserving memory with aging. At baseline, participants provided information about typical dietary intakes related to cognitive function. About one-third claimed they rarely consumed sugar (29%), many (86%) consumed fish once weekly, about half (42%) rarely ate at fast food restaurants, nearly half (43%) consumed less than one serving of red meat and cold cuts weekly, about three-quarters of the group (71%) consumed one or fewer servings of fruits and vegetables daily, most (81%) ate seeds or nuts once or less each week, and about three-quarters (71%) had at least one alcoholic beverage per week.

Four of seven subjects regularly used dietary supplements: four took fish or krill oil, multivitamins, vitamin D, glucosamine, turmeric, probiotics, and prebiotics. Five participants exercised weekly for a total of 30-60 minutes total and walking was the most common form of it.

Dietary Intervention

During the first three weeks, four subjects consumed the two prescribed brain-targeted beverages daily, two others consumed about one and one-half of the two, and one participant consumed one of the two beverages. Those who consumed less than the recommended amount reported to the assigned coach that they found the two beverages over-satiating. Subsequently, compliance with the dietary regimen improved and by week 6, all were consuming the two assigned brain-targeted beverages. Four participants prepared the beverage with water and three used milk (e.g., cow, coconut), and the majority added other foods like peanut butter, berries, syrups, and ice.

Cognitive Test

Cognitive Failures Questionnaire (CFQ)

Each of the summed sub-scale scores improved over time (i.e., scores went down) (Table 1). At week 3, compared to baseline, Total CFQ score was 20% lower; Forgetfulness was 14% lower, Distractibility was 26% lower, and False triggering was 19% lower. At week 6, compared to baseline, each sub-scale score improved more. The Total CFQ score was 36% lower, Forgetfulness was 33% lower, Distractibility was 35% lower, and False triggering was 44% lower. All but one participant experienced improvement in the Total CFQ score.

All 25 individual responses improved over 6 weeks and mimicked the summed sub-scale scores (Table 2). Most (60%) of the responses improved compared to baseline at week 3, and further improvement was observed at week 6 compared to both baseline and week 3. The questions that exhibited the most improvement were for no longer forgetting where to turn off a road (75% lower score); no longer losing track of paying bills and now noticing sign posts (both with 67% lower score); and less forgetfulness of whether a light was turned off or a door locked (50% lower score).

Subjective Memory Questionnaire Responses (SMQ)

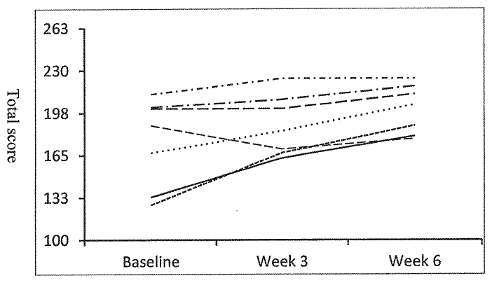

The mean baseline total score for the 33 SMQ questions represented a group that had a modest appreciation of losing memory (175.71±34.44) (Table 3). Three subjects had normal memory (a score of 200 points or more) and four had modest impairment (scores of 100-200) at baseline. Compared to baseline, at week 3, the improvement of the total score was 7% and at week 6, improvement doubled (14%). Those with higher total scores at the beginning of the study (worse cognition) improved most (FIG. 1).

Some individual SMQ questions yielded significant improvements (data not shown). Participants were better able to remember what someone recently told them (P=0.04, week 3 compared to baseline; P=0.005, week 6 compared to baseline); and were able to remember the opening paragraph after completing an article (P=0.02, baseline compared to week 6). Other questions approached near-significance (P>0.05 and <0.1): better able to remember names (baseline to week 3); remembering phone numbers that were just looked up and those used frequently (compared to baseline for both weeks 3 and 6); recalling words (compared to baseline for both weeks 3 and 6); and remembering things that were done last month (compared to baseline for week 6).

Quality of Life Questions

At the end of the study, six of the nine quality of life questions improved (general feeling of wellbeing, fullness, energy, sleep, appearance, and diet quality). The largest improvements between week 6 and baseline were for fullness (50% increase), sleep (18% increase), and appearance and diet quality (both with a 13% increase). Compared to baseline, feeling full increased significantly (P=0.007) at week 6. Mood did not change over time and gastrointestinal symptoms worsened due to one subject having fewer bowel movements and four experiencing more gas, bloating, and bowel movements. Compliance with the dietary regimens were not affected by these gastrointestinal issues.

Discussion

No treatments exist for dementia including Alzheimer's disease, and prevention is the only option to avoid cognitive decline with aging. Diet is the most important prevention therapy and there is much scientific agreement as to what to eat (Gomez-Pinilla F, 2008; Mao X et al, 2019; Mohajeri M H et al, 2015; Morris M C et al, 2015; Shannon O M et al, 2019; Zwilling C E et al, 2019). Paradoxically, most Americans consume a diet that is rich in things that worsen cognition such as processed foods that are low in essential nutrients and rich in sugar, salt, and saturated fats; and avoid nutrient-dense foods that arrest decline (Dietary Guidelines for Americans, 2015). We demonstrated that a dietary intervention with two nutritional therapeutic beverages containing brain-targeted ingredients improved cognitive function and quality of life over six weeks. Participants remained weight stable, which was an objective of the study. It is possible that weight change could have affected cognitive function.

The Cognitive Failures Questionnaire (CFQ) for all sub-scale scores improved: Total CFQ, False triggering, Forgetfulness, and Distractibility. Individual responses improved 14-44% for the sub-scale scores for the 25 questions. Responses for the Subjective Memory Questionnaire (SMQ) were less impressive likely because the baseline scores represented a population that had only a modest memory loss.

The following is the active ingredient matrix in the brain-targeted nutritional therapeutic beverage:
L-Leucine
L-Isoleucine USP
L-Valine
L-Tryptophan USP
L-Histidine Hydrocholoride Monohydrate
L-Lysine Mono HCL Powder
L-Methionine
L-Threonine
L-Cysteine Hydrochloride Monohydrate
L-Phenylalanine USP
L-Tyrosine
L-Arginine
N-Acetyl-L-Cysteine
Acetyl-L-Carnitine Hydrochloride
Phosphatidyl Serine 30%
Quercetin
Alpha Lipoic Acid
Coenzyme Q10 (Ubidecarenone)
Copper Bisglycinate Chelate
Zinc Citrate Dihydrate
Potassium Gluconate
Tripotassium Citrate
Calcium Lactate
Chromium Chloride hexahydrate Milled
Microencapsulated MgO 40%
Manganese Sulfate 1 H2O USP/FCC Powder
Potassium Iodide USP
Dipotassium Phosphate
Selenium L-Methionine Complex 5,000 mcg/g
Molybdenum Trituration
Vit A palmitate
Thiamin Mononitrate
Cyanocobalamin
Riboflavin
Niacinamide Powder
D-calcium Pantothenate
Pyridoxine HCL fine powder
D-Biotin
Folic Acid USP
Microencapsulated Ascorbic Acid
Microencapsulated Vit D 3 Powder
DL a-tocopheryl acetate 50% SD
Vit K1
Choline Bitartrate
Flax Seed
Non-GMO Expeller Pressed Canola Oil
Ferrous Bisglycinate Chelate

Following is an exemplary ingredient list for the brain-targeted nutritional therapeutic beverage consumed per the study protocol:

Nutritional Therapeutic Beverage

| | Single Serving |
|---|---|
| Ingredient | (wt. %) |
| L-Leucine Regular | 1.5654% |
| L-Isoleucine USP | 0.8028% |
| L-Valine | 1.0436% |
| L-Tryptophan USP | 0.1606% |
| L-Histidine Hydrocholoride Monohydrate | 0.5396% |
| L-Lysine Mono HCL Powder | 1.5046% |
| L-Methionine | 0.4014% |
| L-Threonine | 0.6021% |
| L-Cysteine Hydrochloride Monohydrate | 0.2316% |
| L-Phenylalanine USP | 0.5017% |
| L-Tyrosine | 0.5017% |
| L-Arginine | 0.2649% |
| N- Acetyl -L-Cysteine | 1.3042% |
| Acetyl-L-Carnitine Hydrochloride | 0.5968% |
| Phosphatidyl Serine 30% | 1.5814% |
| Quercetin | 0.4658% |
| Alpha Lipoic Acid | 0.1597% |
| Coenzyme Q10 (Ubidecarenone) | 0.0807% |
| Copper Bisglycinate Chelate | 0.0104% |
| Zinc Citrate Dihydrate | 0.0250% |
| Potassium Gluconate | 1.5395% |
| Tripotassium Citrate | 0.7106% |
| Calcium Lactate | 4.0995% |
| Chromium Chloride hexahydrate Milled | 0.0003% |
| Microencapsulated MgO 40% | 1.0872% |
| Manganese Sulfate 1 H2O USP/FCC Powder | 0.0033% |
| Potassium Iodide USP | 0.0001% |
| Dipotassium Phosphate | 2.9958% |
| Selenium L-Methionine Complex | 0.0076% |
| 5,000 mcg/g HFG | |
| Molybdenum Trituration | 0.0008% |
| Microencapsulated Vit A palmitate | 0.0179% |
| Thiamin Mononitrate | 0.0019% |
| Cyanocobalamin | 0.0005% |
| Riboflavin | 0.0015% |
| Niacinamide Powder | 0.0154% |
| D-calcium Pantothenate | 0.0105% |
| Pyridoxine HCL fine powder | 0.0019% |
| D-Biotin | 0.0219% |
| Folic Acid USP | 0.0036% |
| Microencapsulated Ascorbic Acid | 0.1023% |
| Microencapsulated Vit D 3 Powder | 0.0033% |
| DL α-tocopheryl acetate 50% SD | 0.0438% |
| Vit K1 Microencapsulated 1% | 0.0067% |
| Maltodextrin | 0.0138% |
| Choline Bitartrate Coated | 0.7195% |
| Palm Kernel Oil | 0.1281% |
| Oat Fiber | 0.1020% |
| Instant Non Fat Dry Milk Fortified Extra Grade | 14.2326% |
| Whey Protein Concentrate 80 | 17.3954% |
| Purified Sea Salt | 0.1581% |
| Soy Fiber | 3.9535% |
| Natural Vanilla Type Powder | 1.1861% |
| Bitter Blocker | 0.1265% |
| Cocoa Powder Processed with Alkali | 12.6512% |
| Cream Powder 72% | 3.1628% |
| Guar Gum | 1.1861% |
| Flax Seed | 3.1628% |
| Extra Fine Granulated Sugar | 6.0093% |
| Monk Fruit 50 | 0.0633% |
| Stevia | 0.0633% |
| Non-GMO Expeller Pressed Canola Oil | 12.6512% |
| Ferrous Bisglycinate Chelate | 0.0144% |
| Total for Chocolate Shake | 100% |

Consumption of the two brain-targeted beverages daily, as per the protocol, provided two-thirds of the daily requirement for every vitamin and mineral, while being low in ingredients that worsen cognition (i.e., sugar, sodium, and saturated fat). Diets that contain less than 50 g of added sugars, 2,300 mg of sodium, and 20 g of saturated fat are optimal for cognitive health (https://www.fda.gov/food/new-nutrition-facts-label/daily-value-new-nutrition-and-supplement-facts-labels).

The beverages contained brain-targeted ingredients that have been shown to improve cognitive function (Glade M J, 2015; Ho L et al, 2013; Kidd P M, 2005; Richter Y et al, 2013; Schreiber S et al, 2000; Shahripour R B et al, 2014; Shenk J C, 2009). These compounds serve as antioxidants to protect the brain and one, phosphatidylserine, has been shown to improve cognitive function (Glade M J, 2015; Richter Y et al, 2013; Schreiber S et al, 2000). It even has a Qualified Health Claim from the Food and Drug Administration because of its reported efficacy (Hubbard W K, 2004).

The two beverages provided two-thirds of daily AI for choline. Choline is under-consumed unless the diet regularly includes egg yolks, red meats, and seafood (Derbyshire K, 2019). Higher dietary choline intakes, especially as phosphatidylcholine from food or supplements, is associated with better cognitive performance and a lower risk of dementia (Ylilauri M P T et al, 2019).

The finding that the dietary intervention improved the quality of life was not surprising, given that similar improvements were observed in prospective studies with different conditions (e.g., hypertension, type 2 diabetes, and males with reduced libidos) (Bauer K et al, 2019; Morehouse N G et al, 2019; Wijendran V et al, 2019). In these studies, participants consumed similar nutrient-dense foods but without the brain-targeted ingredients.

The diet quality of most Americans is poor, mainly due to lacking essential nutrients and having too many components that increase the risk of chronic disease including dementia (e.g., salt, sugar, and saturated fats) (Dietary Guidelines for Americans, 2015). From baseline dietary questionnaires, the participants had dietary habits that mimicked the American public. The subjects over consumed sugar, frequented fast food restaurants that offer foods rich in salt and saturated fats, and had sub-optimal intakes of fruits, vegetables, seeds, and nuts. This nutrient-poor dietary pattern contains too many calories from sugar (double what is recommended) and fats, especially saturated fats (most people consume 70% more than the recommended amount), and has been shown to increase mortality and the risk of chronic conditions (Li Y et al, 2018; Schwingshackl L et al, 2017).

Brain-targeted dietary interventions have been shown to improve cognitive function in those with mild cognitive impairment as a result of Alzheimer's disease or pre-Alzheimer's (Bredesen D E, 2014, 2018). The dietary program, metabolic enhancement of neurodegeneration, includes a healthy eating program, which emphasizes whole foods (i.e., fruits, vegetables, whole grains), seafood rather than red meats, and limiting processed foods, especially refined carbohydrates. In addition to dietary recommendations, a more personalized approach is provided that includes recommending dietary supplements like vitamins D, and E; minerals like zinc; fish oil to provide omega-3 fatty acids; and others like coenzyme Q10, and alpha lipoic acid. Reversal of cognitive decline was observed in 100 patients, who have undergone this targeted dietary approach (Bredesen D E et al, 2018). Improvement was demonstrated in cognitive function tests, reports from family members, and in some cases, electrophysiology (imaging).

### CONCLUSION

Prevention is the only viable option to arrest cognitive decline with aging, and necessary as no treatments exist for dementia. Most of the evidence supports a role for a heathy, nutrient-rich diet that is low in sugar, sodium, and saturated fats. The participants in this study consumed two brain-targeted beverages that satisfied these criteria and included other bioactive compounds known to enhance cognitive function. After six weeks, improvements were observed in sub-categories of the Cognitive Failures Questionnaire and total scores for Subjective Memory questions. In addition, subjects experienced improvement in quality of life related to general feeling of wellbeing, fullness, energy levels, sleep, and eating better. These promising findings present an easy way to enhance diet and improve cognition.

TABLE 1
Changes in summed sub-scale scores for the Cognitive Failures Questionnaire (CFQ)

| | Total CFQ score | Forgetfulness | Distractibility | False triggering |
|---|---|---|---|---|
| Baseline | 28.43 ± 18.20 | 11.57 ± 5.86 | 9.29 ± 6.21 | 8.14 ± 6.59 |
| Week 3 | 22.86 ± 10.76 | 10.00 ± 2.89 | 6.86 ± 4.14 | 6.57 ± 4.28 |
| vs. Baseline | 20% lower | 14% lower | 26% lower | 19% lower |
| Week 6 | 18.43 ± 11.89 | 7.71 ± 4.92 | 6.00 ± 3.51 | 4.57 ± 4.35 |
| vs. Baseline | 36% lower | 33% lower | 35% lower | 44% lower |

TABLE 2
Cognitive Failures Questionnaire responses to individual questions

| | | Week | Week |
|---|---|---|---|
| Question | Baseline | 3* | 6* |
| Do you read something and find | 1.71 ± | 1.43 ± | 1.14 ± |
| you haven't been thinking about | 1.1 | 0.79 | 0.69 |
| it and must read it again? | | 17% | 33% |
| | | lower | lower |
| Do you find you forget why you | 1.57 ± | 1.43 ± | 1.0± |
| went from one place of the house | 1.13 | 0.78 | 1.00 |
| to the other? | | 9% | 36% |
| | | lower | lower |
| Do you fail to notice signposts | 0.86 ± | 0.57 ± | 0.29 ± |
| on the road? | 1.07 | 0.70 | 0.49 |
| | | 33% | 67% |
| | | lower | lower |
| Do you find you confuse right | 0.86 ± | 0.71 ± | 0.57 ± |
| and left when giving people | 1.21 | 1.25 | 0.79 |
| directions? | | 17% | 33% |
| | | lower | lower |
| Do you bump into people? | 1.00 ± | 1.14 ± | 0.71 ± |
| | 0.79 | 1.25 | 0.76 |
| | | 14% | 29% |
| | | higher | lower |
| Do you find you forget | 1.43 ± | 1.43 ± | 0.71 ± |
| whether you've turned off a | 0.79 | 0.97 | 0.76 |
| light or locked the door? | | No | 50% |
| | | change | lower |
| Do you fail to listen to people's | 1.29 ± | 1.14 ± | 0.86 ± |
| names when you are meeting | 1.25 | 1.07 | 0.90 |
| with them? | | 11% | 33% |
| | | lower | lower |
| Do you say something and | 1.43 ± | 1.00 ± | 0.86 ± |
| realize afterwards that it might | 1.13 | 0.82 | 0.90 |
| be taken as insulting? | | 30% | 40% |
| | | lower | lower |
| Do you fail to hear people | 1.00 ± | 0.71 ± | 0.57 ± |
| speaking to you when you | 1.00 | 0.76 | 0.79 |
| are doing something else? | | 29% | 43% |
| | | lower | lower |
| Do you lose your temper and regret it? | 1.00 ± | 0.43 ± | 0.57 ± |
| | 0.82 | 0.79 | 0.79 |
| | | 57% | 43% |
| | | lower | lower |
| Do you lose track of paying bills? | 0.43 ± | 0.29 ± | 0.14 ± |
| | 0.79 | 0.76 | 0.38 |
| | | 33% | 67% |
| | | lower | lower |
| Do you find you forget which | 0.57 ± | 0.14 ± | 0.14± |
| way to turn on a road you know | 0.98 | 0.38 | 0.38 |
| well but rarely use? | | 75% | 75% |
| | | lower | lower |
| Do you fail to see what you want | 0.57 ± | 0.43 ± | 0.71 ± |
| in a supermarket (although it's there)? | 0.79 | 0.53 | 0.76 |
| | | 25% | 25% |
| | | lower | higher |
| Do you find yourself suddenly | 1.29 ± | 1.00 ± | 0.71 ± |
| wondering whether you've used | 1.11 | 1.15 | 0.76 |
| a word correctly? | | 22% | 44% |
| | | lower | lower |
| Do you have trouble making | 1.14 ± | 1.00 ± | 0.86 ± |
| up your mind? | 0.90 | 0.58 | 0.9 |
| | | 12% | 25% |
| | | lower | lower |
| Do you find you forget an | 0.43 ± | 0.43 ± | 0.29 ± |
| appointment? | 0.79 | 0.79 | 0.49 |
| | | No | 33% |
| | | change | lower |
| Do you forget where you put | 1.43 ± | 1.26 ± | 1.14 ± |
| something like a newspaper or a | 0.98 | 0.49 | 0.69 |
| book? | | 10% | 20% |
| | | lower | lower |
| Do you find you accidentally | 0.71 ± | 0.43 ± | 0.57 ± |
| misplace things like placing the | 1.11 | 0.79 | 0.79 |
| fruit in the cupboard and the | | 40% | 20% |
| bowel in the refrigerator? | | lower | lower |
| Do you daydream when you | 1.71 ± | 1.14 ± | 1.14 ± |
| ought to be listening to something? | 1.38 | 0.69 | 0.90 |
| | | 33% | 33% |
| | | lower | lower |
| Do you find you forget people's names? | 1.86 ± | 1.43 ± | 1.00 ± |
| | 0.69 | 0.96 | 1.00 |
| | | 23% | 46% |
| | | lower | lower |
| Do you start doing one thing at | 2.00 ± | 1.86 ± | 1.57 ± |
| home and get distracted and | 1.29 | 1.35 | 0.98 |
| do something else (unintentionally)? | | 7% | 21% |
| | | lower | lower |
| Do you find you can't quite | 1.71 ± | 1.57 ± | 1.29 ± |
| remember something although | 0.76 | 0.53 | 0.76 |
| it's “on the tip of your tongue”? | | 8% | 25% |
| | | lower | lower |
| Do you find you forget what you | 1.00 ± | 0.57 ± | 0.57 ± |
| came to the store to buy? | 1.00 | 0.79 | 0.79 |
| | | 43% | 43% |
| | | lower | lower |
| Do you drop things? | 1.00 ± | 0.86 ± | 0.57 ± |
| | 1.00 | 0.69 | 0.53 |
| | | 14% | 43% |
| | | lower | lower |
| Do you find you can't think | 0.43 ± | 0.43 ± | 0.43 ± |
| of anything to say? | 0.53 | 0.79 | 0.79 |
| | | No | No |
| | | change | change |

*Compared to Baseline. Scoring goes from 0 (never) to 4 (very often); lower number is better.

TABLE 3
Changes in Subjective Memory questionnaire responses

| | Total score | Mean individual score |
|---|---|---|
| Baseline | 175.71 ± 34.44 | 5.32 ± 1.04 |
| Week 3 | 188.14 ± 23.35 | 5.70 ± 0.71 |
| vs. Baseline | 7% increased | 7% increased |
| Week 6 | 200.57 ± 18.64 | 6.08 ± 0.56 |
| vs. Baseline | 14% increased | 14% increased |

TABLE 4
Changes in quality of life

| Quality of life | | | |
|---|---|---|---|
| questions | Baseline | Week 3 | Week 6 |
| How did you | 3.57 ± 0.98 | 3.43 ± 0.98 | 4.00 ± 0.82 |
| generally feel? | | | 12%* |
| Did you feel full? | 2.86 ± 0.90 | 3.86 ± 1.07\hat{ } | 4.29 ± 0.76 # |
| | | | 50% |
| How was your mood? | 4.14 ± 1.07 | 4.43 ± 0.54 | 4.14 ± 0.69 |
| | | | 0% |
| How was your | 3.57 ± 0.53 | 3.71 ± 0.49 | 3.86 ± 0.38 |
| energy? | | | 8% |
| Did you experience | 4.57 ± 0.53 | 4.14 ± 0.69 | 4.00 ± 0.82 |
| any GI issues?+ | | | −12% |
| How did you sleep? | 3.14 ± 1.21 | 3.57 ± 0.98 | 3.71 ± 0.95 |
| | | | 18% |
| How was your | 3.43 ± 0.53 | 3.57 ± 0.53 | 3.86 ± 0.69 |
| appearance? | | | 13% |
| How was your diet | 3.29 ± 0.95 | 3.57 ± 0.79 | 3.71 ± 0.76 |
| quality? | | | 13% |

*Represents the percentage change from baseline to week 6
\hat{ } P = 0.08 vs. Baseline by paired t-test
# P = 0.007 vs. Baseline by paired t-test
+2 subjects had no symptoms; 1 with fewer bowel movements; and 4 had more gas and bloating, and more bowel movements

The FIGURE shows that the baseline Subjective Memory questionnaire total scores of the participants are all above 100, indicating that no one had a great self-awareness of memory difficulties. Moreover, this FIGURE shows that the total scores increased over time, and that those having lower scores at baseline experienced the most beneficial change.

A number of implementations have been described. Nevertheless, it will be understood that additional modifications may be made without departing from the scope of the inventive concepts described herein, and, accordingly, other examples are within the scope of the following claims.

What is claimed is:

1. A brain-targeted nutritional therapeutic beverage that improves cognitive function by at least 14% and up to 44% including forgetfulness, distractibility, and false triggering.

2. The nutritional therapeutic beverage of claim 1 comprising a mixture of L-Phenylalanine, L-Tyrosine, L-Arginine, N-Acetyl-L-Cysteine (NAC), Acetyl-L-Carnititine, Quercetin, Alpha Lipoic Acid, Coenzyme Q10 (Ubidecarenone).

3. The nutritional therapeutic beverage of claim 2, comprising a mixture of at least Calcium, Magnesium, Manganese, Phosphorus, Potassium, Vitamin C, Vitamin D, Vitamin K, Biotin, Chloride, Chromium, Copper, Folate/folic acid, Molybdenum, Niacin, Pantothenic acid, Riboflavin, Selenium, Sodium, Thiamin, Vitamin A, Vitamin B6, Vitamin B12, Vitamin E, Zinc, Choline, Iodine, Iron, L-Phenylalanine, L-Tyrosine, L-Arginine, N-Acetyl-L-Cysteine (NAC), Acetyl-L-Carnitine, Quercetin, Alpha Lipoic Acid, Coenzyme Q10 (Ubidecarenone).

4. The nutritional therapeutic beverage of claim 3, comprising a mixture of at least L-Leucine, L-Isoleucine, L-Valine, L-Tryptophan, L-Histidine, L-Lysine, L-Methionine, L-Threonine, L-Cysteine, Calcium, Magnesium, Manganese, Phosphorus, Potassium, Vitamin C, Vitamin D, Vitamin K, Biotin, Chloride, Chromium, Copper, Folate/folic acid, Molybdenum, Niacin, Pantothenic acid, Riboflavin, Selenium, Sodium, Thiamin, Vitamin A, Vitamin B6, Vitamin B12, Vitamin E, Zinc, Choline, Iodine, Iron, L-Phenylalanine, L-Tyrosine, L-Arginine, N-Acetyl-L-Cysteine (NAC), Acetyl-L-Carnitine, Quercetin, Alpha Lipoic Acid, Coenzyme Q10 (Ubidecarenone).

5. The nutritional therapeutic beverage of claim 4, further comprising Phosphatidyl Serine.

6. The nutritional therapeutic beverage of claim 4, further comprising the chelated minerals Copper Bisglycinate Chelate, Zinc Citrate Dihydrate, and Potassium Gluconate, and still further comprising Phosphatidyl Serine L-Leucine.

7. The nutritional therapeutic beverage of claim 1, comprising at least the following ingredients and amounts of ingredients:

| | Single Serving |
|---|---|
| Ingredient | (wt. %) |
| L-Leucine | 1.5654% |
| L-Isoleucine | 0.8028% |
| L-Valine | 1.0436% |
| L-Tryptophan | 0.1606% |
| L-Histidine | 0.5396% |
| L-Lysine | 1.5046% |
| L-Methionine | 0.4014% |
| L-Threonine | 0.6021% |
| L-Cysteine | 0.2316% |
| L-Phenylalanine | 0.5017% |
| L-Tyrosine | 0.5017% |
| L-Arginine | 0.2649% |
| N- Acetyl -L-Cysteine | 1.3042% |
| Acetyl-L-Carnitine Hydrochloride | 0.5968% |
| Phosphatidyl Serine | 1.5814% |
| Quercetin | 0.4658% |
| Alpha Lipoic Acid | 0.1597% |
| Coenzyme Q10 (Ubidecarenone) | 0.0807% |
| Copper Bisglycinate Chelate | 0.0104% |
| Zinc Citrate Dihydrate | 0.0250% |
| Potassium Gluconate | 1.5395% |
| Tripotassium Citrate | 0.7106% |
| Calcium Lactate | 4.0995% |
| Chromium Chloride Hexahydrate | 0.0003% |
| Microencapsulated MgO | 1.0872% |
| Manganese Sulfate 1 H2O USP/FCC Powder | 0.0033% |
| Potassium Iodide USP | 0.0001% |
| Dipotassium Phosphate | 2.9958% |
| Selenium L-Methionine Complex | 0.0076% |
| Molybdenum Trituration | 0.0008% |
| Vit A Palmitate | 0.0179% |
| Thiamin Mononitrate | 0.0019% |
| Cyanocobalamin | 0.0005% |
| Riboflavin | 0.0015% |
| Niacinamide | 0.0154% |
| D-calcium Pantothenate | 0.0105% |
| Pyridoxine HCL | 0.0019% |
| D-Biotin | 0.0219% |
| Folic Acid | 0.0036% |
| Microencapsulated Ascorbic Acid | 0.1023% |
| Microencapsulated Vit D 3 Powder | 0.0033% |
| DL α-tocopheryl acetate | 0.0438% |
| Vit K1 | 0.0067% |
| Maltodextrin | 0.0138% |
| Choline Bitartrate | 0.7195% |
| Palm Kernel Oil | 0.1281% |
| Oat Fiber | 0.1020% |
| Dry Milk | 14.2326% |
| Whey Protein Concentrate 80 | 17.3954% |
| Sea Salt | 0.1581% |
| Fiber | 3.9535% |
| Vanilla | 1.1861% |
| Bitter Blocker | 0.1265% |
| Cocoa Powder | 12.6512% |
| Cream Powder | 3.1628% |
| Guar Gum | 1.1861% |
| Flax Seed | 3.1628% |
| Sugar | 6.0093% |
| Monk Fruit | 0.0633% |
| Stevia | 0.0633% |
| Non-GMO Expeller Pressed Canola Oil | 12.6512% |
| Ferrous Bisglycinate Chelate | 0.0144% |
| Total | 100% |
